(19) United States
(12) Patent Application Publication
(10) Pub. No.: US 2009/0143718 A1
(43) Pub. Date: Jun. 4, 2009

(54) PLASMA TREATMENT PROBE

(75) Inventors: Chunqi Jiang, Los Angeles, CA (US); P. Thomas Vernier, Los Angeles, CA (US); Martin A. Gundersen, San Gabriel, CA (US); Timothy Meyers, Missouri City, TX (US); Leslie Lii-Ying Wang, Vancouver (CA); Jorgen Slots, Irvine, CA (US)

Correspondence Address: MCDERMOTT WILL & EMERY LLP, 2049 CENTURY PARK EAST, 38th Floor, LOS ANGELES, CA, 90067-3208, US

(73) Assignee: UNIVERSITY OF SOUTHERN CALIFORNIA, Los Angeles, CA (US)

(21) Appl. No.: 12/271,681

(22) Filed: Nov. 14, 2008

Related U.S. Application Data
(60) Provisional application No. 60/988,259, filed on Nov. 15, 2007.

(51) Int. Cl. A61C 5/02; A61M 37/00

(52) U.S. Cl. 604/23; 433/224

(57) ABSTRACT

A plasma treatment probe may include a hollow, tubular electrode defining an interior region, and a coaxial insulating tube configured to enclose the electrode. The insulating tube may form a gas flow outlet at one end. An outer chamber may enclose the insulating tube and the hollow electrode, and may have a gas inlet for receiving a gas mixture. The hollow electrode may be configured to receive nanosecond electric pulses, while a gas mixture flows from the gas inlet through the interior region of the electrode, so that a non-thermal plasma can be ignited that exits from the gas flow outlet onto a region of a patient's body to medically treat the region.

### CROSS REFERENCES TO RELATED APPLICATIONS

This application claims the benefit of priority under 35 U.S.C. §119(e) from co-pending, commonly owned U.S. provisional patent application Ser. No. 60/988,259 (the '259 provisional application”), entitled “Plasma Dental Probe For Root Canal Sterilization,” filed Nov. 15, 2007. The content of the '259 provisional application is incorporated herein by reference in its entirety as though fully set forth.

### STATEMENT REGARDING FEDERALLY SPONSORED RESEARCH

This work was funded in part by Grant No. F49620-02-1-0073 from the Air Force Office of Scientific Research. The government has certain rights in the invention.

### BACKGROUND

Despite continuing advances in the control of diseases of microbial origin, prevention of post-operative bacterial infection remains a serious challenge for practitioners in a number of medical fields, including but not limited to endodontology.

For example, conventional methods of eliminating bacteria from the root canal system, such as mechanical cleaning, irrigation, application of hypochlorite and other anti-bacterial compounds, result in rates of post-procedure infection that exceed 10%, even though eliminating bacteria from the root canal system is a major component of endodontic treatment.

Laser systems have been shown to reduce bacteria after root canal surgery. However, the use of laser systems pose many challenges to practitioners, due to the significant cost of the delivery of care, the sizeable investment in capital, the cost of system operation and laser safety training, and laser-induced tissue trauma in patients that requiring days to recover.

### SUMMARY

A plasma treatment probe may include a hollow, tubular electrode defining an interior region, and a coaxial insulating tube. The insulating tube may be configured to enclose the hollow electrode therewithin, and may define a gas flow outlet at one end. An outer chamber may enclose the insulating tube and the hollow electrode. The electrode may be configured to receive nanosecond electric pulses, while a gas mixture flows from an inlet of the outer chamber through the interior region of the electrode, so that a non-thermal plasma is ignited. The non-thermal plasma may exit from the gas flow outlet of the plasma probe onto a region of a patient's body, to medically treat the region.

### BRIEF DESCRIPTION OF THE DRAWINGS

The figures depict one or more implementations in accordance with the present disclosure, by way of example only and not by way of limitations. The drawings disclose illustrative embodiments. They do not set forth all embodiments. Other embodiments may be used in addition or instead.

FIG. 1 illustrates an exemplary plasma treatment probe, in accordance with one embodiment of the present disclosure.

FIG. 2 is a schematic flowchart that illustrates a method of treating a patient using a plasma treatment probe, in accordance with one embodiment of the present disclosure.

### DETAILED DESCRIPTION

The present disclosure describes methods and systems for using a non-thermal plasma for medical treatment purposes, which include but are not limited to root canal sterilization, dentin tubules sterilization, cleaning of dental and gum surfaces during oral surgery, and wound disinfection. The non-thermal plasma is generated from a plasma treatment probe that has a hollow electrode geometry. The non-thermal plasma can initiate and enhance bactericidal reactions without the need for elevated gas temperature. The plasma can be touched by bare hands without causing heating or painful sensation.

In overview, a plasma probe system may include a plasma treatment probe, a high voltage pulse generator, and a gas flow system. The gas flow system may be configured to delivers pre-mixed gases, i.e. a gas mixture, in a controllable and detectable manner to the plasma treatment probe. The gas flow system may include instruments or devices for controlling and monitoring gas flow, such as a flow meter or a mass flow controller. The flow meter may have a flow rate of about 1000 SCCM up to about 20000 SCCM.

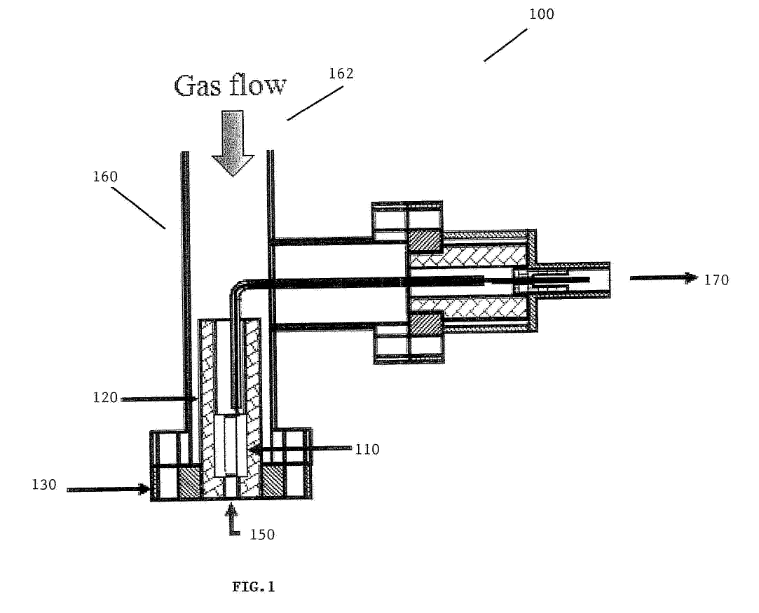

FIG. 1 illustrates an exemplary plasma treatment probe 100, in accordance with one embodiment of the present disclosure. The plasma treatment probe 100 is a room-temperature atmospheric plasma device that can be driven by several kV, hundreds of nanosecond electric pulses. When a flow of a gas mixture is induced, as further described below, the plasma treatment probe may produce a room temperature, pencil-like plasma plume in ambient atmosphere. This plasma plume can be used for many medical applications, including but not limited to: root canal and dentin tubules sterilization after endodontic treatment; cleaning dental surfaces during dental and oral surgery procedures in general; and disinfecting wounds.

The plasma treatment probe 100 employs a coaxial tubular design for the electrodes, and is driven by electric pulses of 100 nanoseconds (or less), which may be generated by a high voltage pulse generator. This design provides a design free of electromagnetic noise, safe to operate, and efficient in electric energy delivery.

The plasma treatment probe 100 shown in FIG. 1 has a hollow-electrode geometry. In overview, the plasma treatment probe 100 includes: a hollow, tubular electrode 110; a coaxial insulating tube 120 that is configured to enclose the electrode therewithin; and an outer chamber 160 that is configured to enclose the insulating tube and the hollow electrode therewithin. The outer chamber 160 may have a gas inlet 162 configured to receive a gas mixture, e.g. from a gas flow system.

In one embodiment, the central electrode 110 may be a high voltage metallic electrode. The electrode 110 may be formed of a variety of metallic materials, including without limitation brass or stainless steel. In one exemplary embodiment, the central electrode 110 may have an inner diameter of about 3 millimeters, an outer diameter of about 6.35 millimeters, and a length of about 12.7 mm. Other embodiments may use central hollow electrodes having different dimensions.

The hollow electrode 110 may configured to receive nanosecond electric pulses, while the gas mixture flows through the interior region of the electrode, so that a non-thermal plasma is ignited.

The high voltage hollow electrode 110 may be enclosed within an isolating tube 120 that is coaxial with the hollow electrode. An outer chamber 160 may enclose the hollow electrode and the insulating tube in a gas tight configuration. A portion of the chamber 160 may be a grounded flange, shown with reference numeral 130 in FIG. 1. In one exemplary embodiment, the grounded flange 130 may have an inner diameter of about 12.7 millimeters, and an outer diameter of about 33.8 millimeters. In one embodiment, the grounded flange 130 may be a Conflat flange made of stainless steel.

In one embodiment, the insulating tube 120 may have an inner diameter of about 6.35 millimeters to accommodate the central metal electrode 110, and a length of about 38 millimeters. One end of the insulating tube 120 may be an exit aperture that functions as a gas flow outlet 150 of the plasma probe 100. In one embodiment, the gas flow outlet 150 may have a length of about five millimeters, and an inner diameter of about three millimeters. The insulating tube 120 may be made of a variety of insulator materials, including without limitation ceramic. The insulating tube 120 may separate and isolate the inner high voltage electrode 110 from the outside air, and from the grounded flange 130.

The plasma probe 100 may be made gas-tight, for example by copper gaskets or Torr-seal glue, to ensure that the gas mixture only exits through the exit aperture or gas flow outlet 150.

The nanosecond electric pulses may be generated by a high voltage pulse generator. A custom-designed, inductive adder-based high voltage pulse generator may be used that is capable of generating up to 10 kV, about 50-100 nanosecond pulses at a rate from single shot to 3 kHz.

These high voltage, nanosecond electric pulses may be delivered through standard coaxial SHV (safe high voltage) connections. High voltage insulated wires may be used to deliver the electric pulses from the SHV connection to the central hollow electrode 110.

A pencil-like, non-thermal plasma plume, which may be about two to three centimeters long, may be formed at the exit aperture or gas outlet 150, pointing away from the high voltage electrode 110, when intense nanosecond electric pulses are applied to the hollow metal electrode 110 while a gas mixture flows through the interior region of the hollow electrode 110. In one embodiment, the gas mixture may be a pre-mixed He/(1%)O₂. The non-thermal plasma may exit from the nozzle at a flow rate of about 1˜10 std. L/min.

When applying the plasma treatment probe 100 to root canal surfaces, the plasma plume (generated by the plasma probe) may substantially eliminate the bacteria within the root canal and the dentin tubules.

A first version of plasma treatment probe 100 has been designed and tested with different organisms including Staphylococcus, Streptococcus, and Bacillus atrophaeus for their growth inhibition. In preliminary experiments, substantially 100% killing of test organisms on nutrient agar plates was observed.

In dentistry, the plasma treatment probe 100 can be used for endodontic and periodontal treatment, including but not limited to root canal disinfection, tooth cleaning, cavity disinfection, and periodontal disease prevention. In addition, the plasma treatment probe 100 may be used for wound disinfection, implant disinfection, and disinfection for fungus-related topical diseases. The plasma treatment probe 100 may be particularly useful in treating areas that are difficult to reach, e.g. small cracks, holes, and on-site biomedical device sterilization.

In operation, a method of root canal sterilization may include: receiving a gas mixture from a gas flow system, at a gas inlet of a plasma dental probe; generating a non-thermal plasma by applying nanosecond electric pulses to a hollow metallic electrode within the plasma dental probe, while the gas mixture is flowing through an interior region of the hollow electrode; and delivering the non-thermal plasma from a gas outlet of the plasma dental probe onto the root canal to sterilize the root canal. The nanosecond electric pulses may have a duration of about 50 to 100 nanoseconds, and an intensity up to about 10 kV.

The plasma probe system described above does not require any harmful gases or liquids. With noble gases (i.e. helium) as buffer, and mixed with low-percentage oxygen (<1%), the bactericidal effect is only initiated with the plasma ignition. Compared to conventional methods, “cold” (or non-thermal) plasma treatment of the root canal system offers a painless, safe, effective, and simple procedure for root canal sterilization. Compared to near infrared laser irradiation for root canal sterilization, the “cold” plasma plume employs enhanced chemistry for bacteria elimination. The heat generated from the plasma is minimum, and does not cause any burning in tissues. Moreover, the plasma treatment probe 100 is simple, low cost, compact, and easy to operate and maintain.

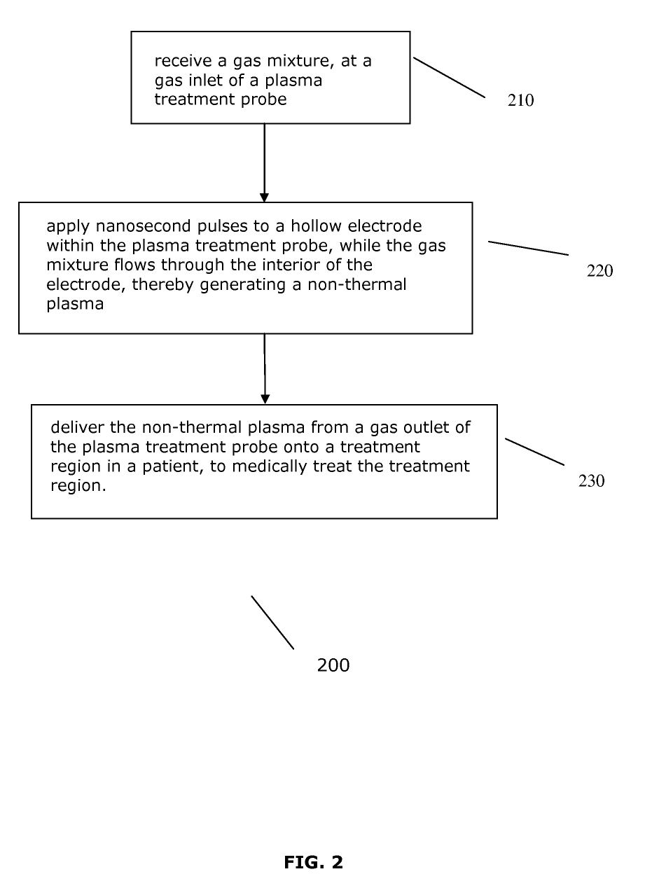

FIG. 2 is a schematic flowchart that illustrates a method 200 of medically treating a patient using a plasma treatment probe, in accordance with one embodiment of the present disclosure. The method 200 may include an act 210 of receiving a gas mixture from a gas flow system, at a gas inlet of a plasma treatment probe. The method 200 may further include an act 210 of generating a non-thermal plasma by applying nanosecond electric pulses to a hollow metallic electrode within the plasma treatment probe, while the gas mixture is flowing through an interior region of the hollow electrode. The method 200 may further include an act 220 of delivering the non-thermal plasma from a gas flow outlet of the plasma treatment probe onto a treatment region in the patient's body, to medically treat the treatment region.

In sum, methods and systems have been described for generating and delivering a non-thermal plasma that can disinfect and sterilize root canal systems, wounds, and other treatment regions of a patient's body, in a painless, safe, effective, and inexpensive manner.

Various changes and modifications may be made to the above described embodiments. The components, steps, features, objects, benefits and advantages that have been discussed are merely illustrative. None of them, nor the discussions relating to them, are intended to limit the scope of protection in any way. Numerous other embodiments are also contemplated, including embodiments that have fewer, additional, and/or different components, steps, features, objects, benefits and advantages. The components and steps may also be arranged and ordered differently.

The phrase “means for” when used in a claim embraces the corresponding structures and materials that have been described and their equivalents. Similarly, the phrase “step for” when used in a claim embraces the corresponding acts that have been described and their equivalents. The absence of these phrases means that the claim is not limited to any of the corresponding structures, materials, or acts or to their equivalents.

Nothing that has been stated or illustrated is intended to cause a dedication of any component, step, feature, object, benefit, advantage, or equivalent to the public, regardless of whether it is recited in the claims.

In short, the scope of protection is limited solely by the claims that now follow. That scope is intended to be as broad as is reasonably consistent with the language that is used in the claims and to encompass all structural and functional equivalents.

What is claimed is:

1. A plasma treatment probe, comprising:
a hollow, tubular electrode defining an interior region;
a coaxial insulating tube configured to enclose the electrode, one end of the insulating tube defining a gas flow outlet; and
an outer chamber configured to enclose the insulating tube and the hollow electrode, the outer chamber having a gas inlet for receiving a gas mixture;
wherein the electrode is configured to receive nanosecond electric pulses, while a gas mixture flows from the gas inlet through the interior region of the electrode, so that a non-thermal plasma can be ignited that exits from the gas flow outlet onto a region of a patient's body to medically treat the region.

2. The plasma treatment probe of claim 1,
wherein the nanosecond electric pulses have a duration of about 50 to 100 nanoseconds, and an intensity up to about 10 kV.

3. The plasma treatment probe of claim 1, wherein the tubular electrode comprises a metallic material.

4. The plasma treatment probe of claim 3, wherein the metallic material comprises one of: brass and stainless steel.

5. The plasma treatment probe of claim 1,
wherein the hollow electrode has an inner diameter of about 3 millimeters, an outer diameter of about 6.35 millimeters, and a length of about 12.7 millimeters.

6. The plasma treatment probe of claim 1,
wherein the insulating tube has an inner diameter of about 6.35 millimeters and a length of about 38 millimeters.

7. The plasma treatment probe of claim 1, wherein the gas flow outlet has an inner diameter of about 3 millimeters and a length of about 5 millimeters.

8. The plasma treatment probe of claim 1,
wherein the outer chamber is configured to enclose the insulating tube and the hollow electrode in a gas tight configuration, and wherein a portion of the chamber is a grounded flange.

9. The plasma treatment probe of claim 8, wherein the grounded flange has an inner diameter of about 12.7 millimeters, and an outer diameter of about 33.8 millimeters.

10. The plasma treatment probe of claim 8,
wherein the insulating tube is configured to shield the tubular electrode from air and from the grounded flange.

11. The plasma treatment probe of claim 8, wherein the gas mixture comprises helium and oxygen.

12. The plasma treatment probe of claim 1, wherein the insulating tube comprises a ceramic material.

13. The plasma treatment probe of claim 8, wherein the outer grounded chamber comprises a stainless steel material.

14. A plasma treatment system, comprising:
a plasma treatment probe including a hollow inner electrode enclosed within an outer coaxial insulating tube, the hollow electrode defining an interior region; and
a pulse generator configured to generate nanosecond electric pulses, and to deliver the electric pulses to the electrode while a gas mixture flows through the interior region of the electrode, so that a non-thermal plasma is generated that can exit from a gas flow outlet of the insulating tube onto a region of a patient's body to medically treat the region.

15. The plasma treatment system of claim 14, wherein the plasma treatment probe further comprises an outer chamber that contains the electrode and the insulating tube, the outer chamber having a gas inlet.

16. The plasma treatment system of claim 15, further comprising a gas flow system configured to generate the gas mixture and to controllably deliver the gas mixture into the gas inlet of the plasma treatment probe.

17. The plasma treatment system of claim 16,
wherein the gas flow system comprises a device for controlling and monitoring flow of the gas mixture into the gas inlet of the plasma probe; and
wherein the device comprises one of:
a gas flow meter; and a mass flow controller.

18. A method of root canal sterilization, comprising:
receiving a gas mixture from a gas flow system, at a gas inlet of a plasma probe;
generating a non-thermal plasma by applying nanosecond electric pulses to a hollow metallic electrode within the plasma probe, while the gas mixture is flowing through an interior region of the hollow electrode; and
delivering the non-thermal plasma from a gas outlet of the plasma probe onto the root canal to sterilize the root canal.

19. The method of claim 18,
wherein the nanosecond electric pulses have a duration of about 50 to 100 nanoseconds, and an intensity up to about 10 kV.

20. A method of medically treating a patient, the method comprising:
receiving a gas mixture from a gas flow system, at a gas inlet of a plasma probe;
generating a non-thermal plasma by applying nanosecond electric pulses to a hollow metallic electrode within the plasma probe, while the gas mixture is flowing through an interior region of the hollow electrode; and
delivering the non-thermal plasma from a gas outlet of the plasma probe onto a treatment region in the patient's body, to medically treat the treatment region.
